(19) United States
(12) Patent Application Publication
(10) Pub. No.: US 2025/0120635 A1
(43) Pub. Date: Apr. 17, 2025

(54) URINE FLOW DATA PROCESSING METHOD AND SYSTEM

(71) Applicants: INVENTEC APPLIANCES CORP., NEW TAIPEI CITY (TW); INVENTEC APPLIANCES (NANCHANG) CORPORATION, JIANGXI (CN)

(72) Inventor: LILIAN LUO, JIANGXI (CN)

(21) Appl. No.: 18/659,550

(22) Filed: May 9, 2024

(30) Foreign Application Priority Data
Oct. 13, 2023 (CN) 2023113303158

(51) Int. Cl. A61B 5/20; A61B 5/00

(52) U.S. Cl. CPC A61B 5/208; A61B 5/7278

(57) ABSTRACT

A urine flow data processing method includes the following steps of: obtaining the urine volume data measured by a uroflowmeter, wherein the urine volume data comprises a first weight at a first time and a second weight at a second time; determining whether the difference between the first weight and the second weight is larger than a presetting threshold value; generating an updated second weight corresponding to the second time according to the first weight, the second weight, and a third weight at a third time when the difference between the first weight and the second weight is larger than the presetting threshold value, and replacing the second weight with the updated second weight to generate an updated urine volume data; and analyzing the updated urine volume data to generate a urine flow chart.

### BACKGROUND OF THE INVENTION

### 1. Field of the Invention

The present invention relates to a data processing method and system, and more particularly, to apply in urine flow data processing method and system with uroflowmeter.

### 2. Description of the Prior Art

Urine examination is one of the common methods to evaluate body healthy status, and using urinary flow testing is one of the inspection items in the urine examination. Urine flow testing is convenient, easy, non-invasive and non-contact test which can further assess whether a patient's urinary system is normal. The urine flow testing device has been widely used in medical institutions for examination. Medical personnel can use curve chart measured by uroflowmeters to determine whether the patient is suffering from urinary diseases such as prostate cancer, chronic prostatitis, urethral stricture, chronic cystitis, urinary tract infection, and urinary dysfunction.

During the process of urinary flow testing, a patient will urinate urine into the container of the uroflowmeter, and the weight sensor of the uroflowmeter will measure the weight of the container at the same time to record the weight difference of the patient's urine to generate urine output data. Then, the urine output data is analyzed to generate a urinary flow chart for evaluation by medical personnel.

However, the error data caused by jitter phenomenon during urinating will make the weight sensor measure abnormal weight data. In detail, too large or too small weight data is not the real urination data of the patient. When the urine volume data is analyzed, the error data will also be analyzed together, and leads to generate the urinary flow chart, which fails to match the actual situation, thereby reducing the correctness and accuracy.

### SUMMARY OF THE INVENTION

Therefore, the present invention provides a urine flow data processing method to solve the problems with the prior art.

In one embodiment of the present invention, the urine flow data processing method comprises the following steps of: obtaining a urine volume data measured by a uroflowmeter, wherein the urine volume data comprises a first weight at a first time and a second weight at a second time, and the second time is later than the first time; determining whether the difference between the first weight and the second weight is larger than a presetting threshold value; generating an updated second weight corresponding to the second time according to the first weight, the second weight, and a third weight at a third time when the difference between the first weight and the second weight is larger than the presetting threshold value, and replacing the second weight with the updated second weight to generate an updated urine volume data, wherein the third time is later than the second time; and analyzing the updated urine volume data to generate a urine flow chart.

Wherein the step of generating the updated second weight corresponding to the second time according to the first weight, the second weight, and a third weight when the difference between the second weight and the third weight is larger than the presetting threshold value, and replacing the updated second weight with the second weight to generate the updated urine volume data of the step, further comprising the following steps of: adding the first weight and the third weight together and then dividing the two to generate the updated second weight when the second weight is larger than or less than the first weight and the third weight, and replacing the second weight with the updated second weight to generate the updated urine volume data.

When the second weight is less than the first weight and the third weight is less than the second weight, replacing the second weight and the third weight with the first weight to generate the updated urine volume data. When the difference between the updated third weight and the fourth weight is larger than the presetting threshold value, generating an updated third weight corresponding to the third time according to the second weight, the third weight and a fourth weight at a fourth time, and replacing the third weight with the updated third weight to generate the updated urine volume data, wherein the fourth time is later than the second time.

Wherein, the presetting threshold value is between 1 g and 1.5 g. The present invention also provides a urine flow data processing system to solve the problems with the prior art. In one embodiment of the present invention, the urine flow data processing system comprises a data retrieval unit, a comparison calculation unit, and a data analysis unit. The data retrieval unit is configured to obtain the urine volume data measured by a uroflowmeter. The comparison calculation unit is connected to the data retrieval unit and pre-stored a presetting threshold value. The comparison calculation unit is configured to calculate the difference between the first weight and the second weight. When the difference is larger than the presetting threshold value, the comparison calculation unit generates the updated second weight corresponding to the second time according to the first weight, the second weight, and a third weight at a third time, and replaces the second weight with the updated second weight to generate an updated urine volume data. Wherein the third time is later than the second time. The data analysis unit is connected to the comparison calculation unit and configured to analyze the updated urine volume data to generate a urine flow chart.

Wherein, the comparison calculation unit further calculates the difference between the second weight and the third weight. When the difference between the first weight and the second weight and the difference between the second weight and the third weight are both larger than the presetting threshold value, and the second weight is also larger than or less than the first weight and the third weight, the comparison calculation unit adds the first weight and the third weight together, and then divides the two to generate an updated second weight.

When the second weight is less than the first weight and the third weight is less than the second weight, the comparison calculation unit replaces the second weight and the third weight with the first weight. When the difference is larger than the presetting threshold value, the comparison calculation unit generates an updated third weight corresponding to the third time according to the updated second weight, the third weight and a fourth weight of a fourth time, and the comparison calculation unit replaces the third weight with the updated third weight to generate the updated urine volume data. Wherein, the fourth time is later than the third time.

In summary, the urine flow data processing method of the present invention can accurately determine the abnormal measurement data caused by the jitter phenomenon, and further reasonably adjust the abnormal measurement data according to the trend of the urine volume data. Furthermore, the urine flow data processing method of the present invention can iteratively determine and adjust urine volume data one by one in ascending order of time to remove abnormal data with jitter phenomenon, so that the urine flow chart generated from the urine volume data can be more consistent with the actual situation, so as to improve the correctness and accuracy.

### BRIEF DESCRIPTION OF THE APPENDED DRAWINGS

FIG. 1 is a flow chart illustrating a urine flow data processing method according to an embodiment of the present invention.

FIG. 2 is a curve chart illustrating the urine volume data according to an embodiment of the present invention.

FIG. 3 is a flow chart illustrating the urine flow data processing method according to an embodiment of the present invention.

FIG. 4A is a curve chart illustrating the urine volume data according to an embodiment of the present invention.

FIG. 4B is a curve chart illustrating the urine volume data according to an embodiment of the present invention.

FIG. 4C is a curve chart illustrating the urine volume data according to an embodiment of the present invention.

FIG. 5 is a flow chart illustrating the urine flow data processing method according to an embodiment of the present invention.

FIG. 6 is a functional block diagram illustrating the urine flow data processing system according to an embodiment of the present invention.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention, wherein the specific methods, devices, conditions, materials and the like are not limited to the embodiments of the present invention or corresponding embodiments. Moreover, the devices in the figures are only used to express their corresponding positions and are not drawing according to their actual proportion.

In the description of this specification, the description with reference to the terms “a specific embodiment”, “another specific embodiment” or “parts of specific embodiments” etc. means that the specific feature, structure, material or feature described in conjunction with the embodiment include in at least one embodiment of the present invention. In this specification, the schematic representations of the above-mentioned terms do not necessarily refer to the same embodiment. Moreover, the described specific features, structures, materials or characteristics can be combined in any one or more embodiments in a suitable manner.

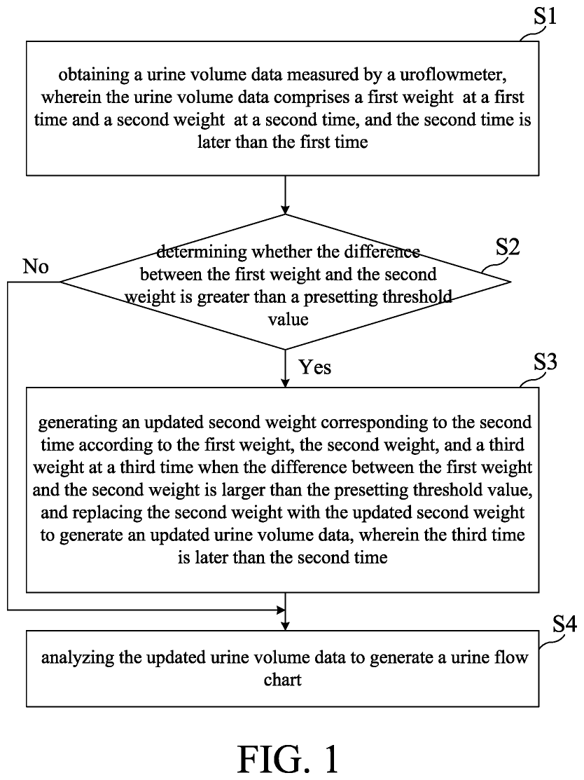

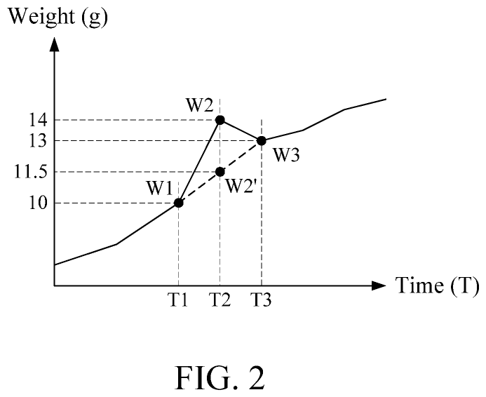

Please refer to FIG. 1 and FIG. 2. FIG. 1 is a flow chart illustrating a urine flow data processing method according to an embodiment of the present invention. FIG. 2 is a curve chart illustrating the urine volume data according to an embodiment of the present invention. As shown in FIG. 1, the urine flow data processing method of the present invention comprises the following steps of: step S1: obtaining a urine volume data measured by a uroflowmeter, wherein the urine volume data comprises a first weight at a first time and a second weight at a second time, and the second time is later than the first time; step S2: determining whether the difference between the first weight and the second weight is larger than a presetting threshold value; step S3: generating an updated second weight corresponding to the second time according to the first weight, the second weight, and a third weight at a third time when the difference between the first weight and the second weight is larger than the presetting threshold value, and replacing the second weight with the updated second weight to generate an updated urine volume data, wherein the third time is later than the second time; and step S4: analyzing the updated urine volume data to generate a urine flow chart.

The urine flow data processing method of the present invention will preprocess the urine volume data measured by the uroflowmeter, adjust the abnormal or unreasonable data into normal range, and analyze the data. In step S1, the urine volume data is obtained by continuously measuring the weight in the container of the uroflowmeter through the weight sensor of the uroflowmeter. Therefore, the urine volume data includes multiple times and multiple weights, and each weight corresponds to a time. In practice, the weight sensor of the uroflowmeter can record the accumulated weight data at a time interval, and the time interval can be 0.05 seconds, which means the weight sensor records once every 0.05 seconds, but is not limited thereto. In practical applications, the time interval can be determined according to design or requirements.

In step S2, the presetting threshold value is configured to be regarded as a basis for determining whether the urine volume data contains abnormal or unreasonable weights. In practice, the presetting threshold value is between 1 g and 1.5 g, but is not limited thereto. The presetting threshold value can be determined based on the accuracy of the weight sensor of the uroflowmeter, the flow rate range to be measured, or the error tolerance range of urine volume data determined by experience or experts. In practical applications, since urine or liquid is usually continuously injected into the container of the uroflowmeter, and the weight sensor of the uroflowmeter also continuously measures the accumulated weight data at short time intervals, the urine volume curve formed by the urine volume data can be a continuous and smooth curve.

In the present embodiment, the urine flow data processing method of the present invention determines whether the urine volume data measured by a uroflowmeter needs to be adjusted based on the difference between the weights corresponding to two adjacent times and the preset threshold. In practice, when the difference between the weights at two adjacent times is less than or equal to the preset threshold, the difference is an erroneous data of the weight sensor but still in the normal weight range, the weights corresponding to the two adjacent times do not need to be preprocessed. On the contrary, when the difference between the weights at two adjacent times is larger than the preset threshold, the difference is caused by the jitter phenomenon but not the actual weight of urine or liquid. At this time, the weights corresponding to the two adjacent times need to be preprocessed.

As shown in FIG. 2, the urine volume data includes a first weight W1 at a first time T1, a second weight W2 at a second time T2, and a third weight W3 at a third time T3. The second time T2 is later than the first time T1 and the third time T3 is later than the second time T2. The first weight W1, the second weight W2 and the third weight W3 can be illustrated to form a urine curve (shown as a solid line in FIG. 2). In practical applications, calculating the difference between the second weight W2 and the first weight W1 is based on the first weight W1 at the first time T1. When the difference between the second weight W2 and the first weight W1 is less than the presetting threshold value, the second weight W2 does not need to be adjusted and can be used directly; when the difference between the second weight W2 and the first weight W1 is larger than the presetting threshold value, the second weight W2 needs to be adjusted.

In step S3, when the second weight W2 needs to be adjusted, an updated second weight W2′ will be generated according to the adjacent time (such as the first time T1 and the third time T3) to obtain each weight corresponded to the adjacent time (such as the first weight W1 and the third weight W3). In practice, the updated second weight W2′ is the value obtained by adding the first weight W1 and the third weight W3 and dividing it by two. Then, the second weight W2 is replaced with the updated second weight W2′ at the second time T2 to generate the updated urine volume data. For example, when the first weight W1 is 10 g, the second weight W2 is 14 g, the third weight W3 is 12 g, and presetting threshold value is 1.5 g. The second weight W2 is determined as jitter phenomenon. At this time, the updated second weight W2′ is calculated to (10 g+12 g)/2=11 g and replaces the original weight (the second weight W2). Then, the updated urine volume data will be a continuous and smooth urine volume curve (shown as the dotted line in FIG. 2).

In step S4, the updated urine volume data can be analyzed and calculated through the filtering algorithm to generate the urine flow chart. In practice, the filtering algorithm can be band-pass filtering algorithm, high-pass filtering algorithm, low-pass filtering algorithm, average filtering algorithm, median filtering algorithm, Kalman filtering algorithm, etc., but is not limited thereto. Since the updated urine volume data does not include erroneous data caused by jitter phenomenon, that is to say, erroneous data will not be included in the analysis and calculation of the filtering algorithm.

Therefore, the urine flow data processing method of the present invention can pre-determine the erroneous data caused by jitter phenomenon after measuring the urine volume data, and adjust abnormal or unreasonable data to update the urine volume data without the erroneous data caused by jitter phenomenon.

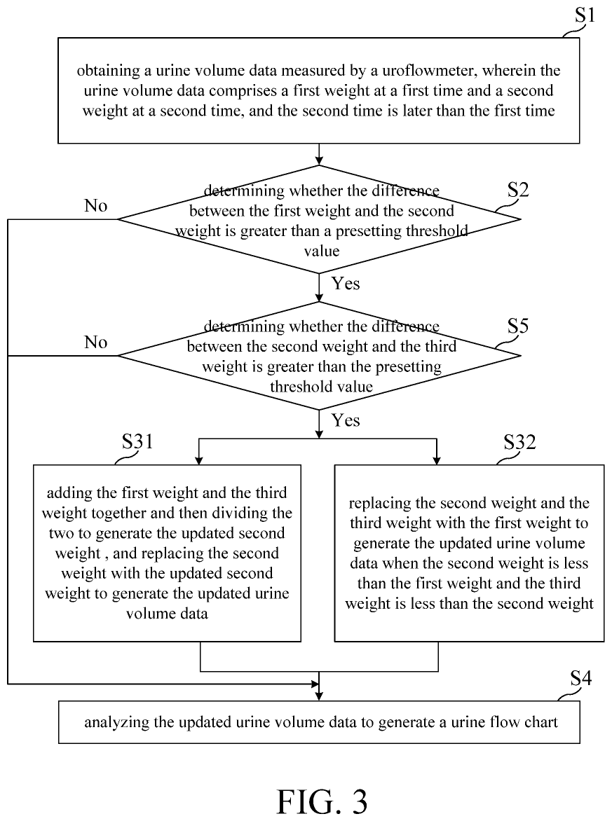

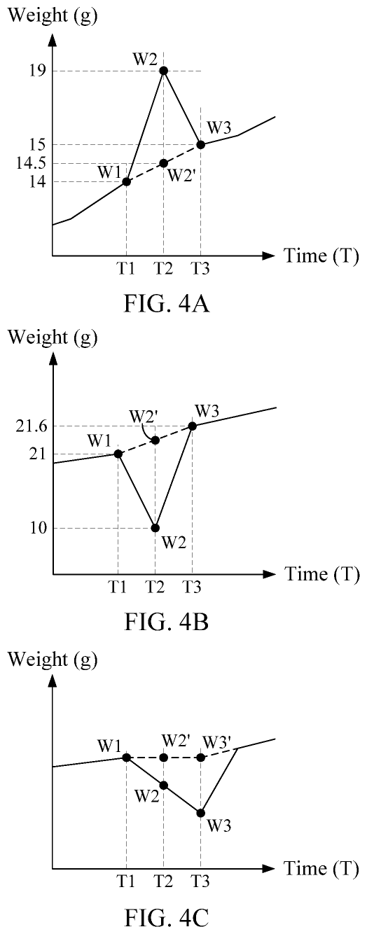

Please refer to FIG. 3, FIG. 4A, FIG. 4B and FIG. 4C. FIG. 4A is a curve chart illustrating the urine volume data according to an embodiment of the present invention. FIG. 4B is a curve chart illustrating the urine volume data according to an embodiment of the present invention. FIG. 4C is a curve chart illustrating the urine volume data according to an embodiment of the present invention. As shown in FIG. 3, in the present embodiment, the urine flow data processing method further comprises the following steps of: step S5: determining whether the difference between the second weight and the third weight is larger than the presetting threshold value; when the result is yes, then the step S3 of FIG. 1 further includes: step S31: adding the first weight and the third weight together and then dividing the two to generate the updated second weight, and replacing the second weight with the updated second weight to generate the updated urine volume data.

In practice, since the erroneous data caused by the jitter phenomenon which is measured by the weight sensor of the uroflowmeter is mostly a momentary value. As shown in FIG. 3, the urine flow data processing method of the present invention will not only determine whether the difference between the first weight and the second weight is larger than the presetting threshold value (step S2), but also determine whether the difference between the second weight and the third weight is larger than the presetting threshold value (step S5). Namely, when the two differences between the above-mentioned values are both larger than a presetting threshold value, the second weight of the second time is regarded as the erroneous data caused by the jitter phenomenon. It should be noted that step S5 is executed after step S2 in FIG. 3, but in practice, step S2 and step S5 can also be executed at the same time.

Next, there are different situations which are illustrated in the urine volume data curves will be described in detail below. FIG. 4A shows a situation that the second weight is adjusted by decreasing. As shown in FIG. 4A, the second weight W2 is larger than the first weight W1 and the third weight W3, and both of the differences (the difference between the first weight and the second weight and the difference between the second weight and the third weight) are larger than the presetting threshold values (the presetting threshold value is 1 g). Namely, the second weight W2 is the erroneous data that became heavier caused by the jitter phenomenon. At this time, the updated second weight W2′ is calculated to be (14 g+15 g)/2=14.5 g, and replaces the original value (the second weight W2), so as to eliminate abnormal data.

Next, FIG. 4B shows a situation that the second weight is adjusted by increasing. As shown in FIG. 4B, the second weight W2 is less than the first weight W1 and the third weight W3, and both of the difference (the difference between the first weight and the second weight and the difference between the second weight and the third weight) are larger than the presetting threshold value (the presetting threshold value is 1 g). Namely, the second weight W2 is the erroneous data that became lighter caused by the jitter phenomenon. At this time, the updated second weight W2′ is calculated to be (21 g+21.6 g)/2=21.3 g, and replaces the original value (the second weight W2), so as to eliminate abnormal data and generate a continuous and smooth curve. Therefore, the urine flow data processing method of the present invention is able to accurately identify the jitter phenomenon and adjust the urine volume data.

Furthermore, the urine flow data processing method of the present invention can determine erroneous data caused by the jitter phenomenon, but also further determine other abnormal situation. As shown in FIG. 3, the urine flow data processing method further comprises the following steps of: step S32: replacing the second weight and the third weight with the first weight to generate the updated urine volume data when the second weight is less than the first weight and the third weight is less than the second weight. Next, please refer to FIG. 4C, FIG. 4C shows a situation that the weight is continuous decreasing. As shown in FIG. 4C, the second weight W2 is less than the first weight W1 and the third weight W3 is less than the second weight W2, and both of the difference (the difference between the first weight and the second weight and the difference between the second weight and the third weight) are larger than the presetting threshold value (the presetting threshold value is 1 g). Namely, the second weight W2 and the third weight W3 are the erroneous data that have become lighter for long time by the jitter phenomenon. At this time, the value of the first weight W1 will be the updated second weight W2′ and the updated third weight W3′, and replaces the second weight W2 and the third weight W3, so as to avoid the situation when the weight of the next moment is less than the weight of the previous moment, and improve the accuracy of subsequent analysis.

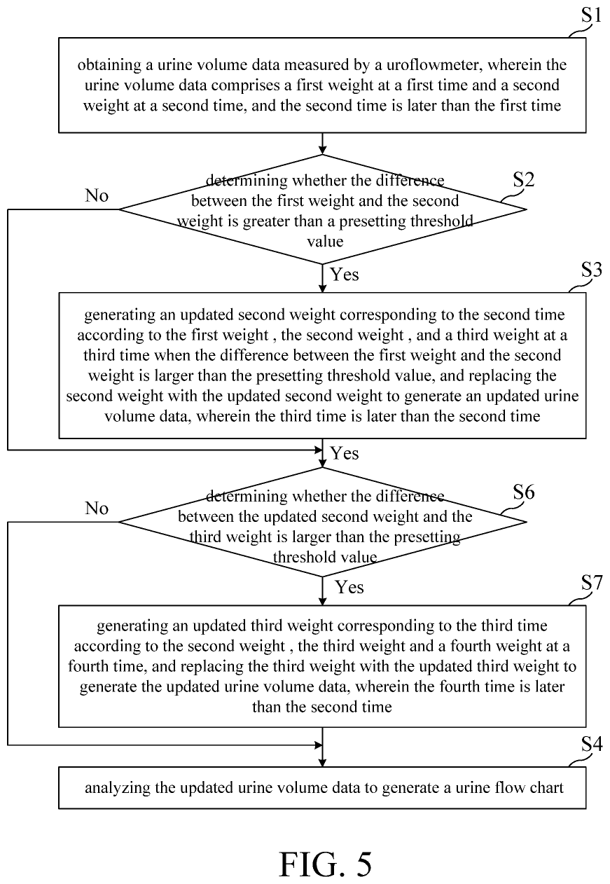

Please refer to FIG. 5. FIG. 5 is a flow chart illustrating the urine flow data processing method according to an embodiment of the present invention. In addition, FIG. 5 is a further step flow of FIG. 1. As shown in FIG. 5, in the present embodiment, after step S3 is executed, the urine flow data processing method further comprises the following steps of: step S6: determining whether the difference between the updated second weight and the third weight is larger than the presetting threshold value; and step S7: generating an updated third weight corresponding to the third time according to the second weight, the third weight and a fourth weight at a fourth time, and replacing the third weight with the updated third weight to generate the updated urine volume data, wherein the fourth time is later than the second time.

The urine flow data processing method of the present invention can further determine and adjust the urine volume data iteratively. In practice, when the second weight is determined as the jitter phenomenon and will replace the second weight with the updated second weight, the urine flow data processing method of the present invention will be further determined whether there is an abnormal data between the updated second weight and the third weight. In step S6, the difference between the updated second weight and the third weight is calculated based on the updated second weight of the second time. When the difference between the updated second weight and the third weight is larger than the presetting threshold value, the third weight at the third time also needs to be adjusted. In step S7, when the third weight needs to be adjusted, an updated third weight W3′ will be generated according to the adjacent time (such as the second time T2 and the fourth time T4) to obtain each weight corresponded at adjacent time (such as the updated second weight W2′ and the fourth weight W4). In practice, the updated third weight W3′ is obtained by adding the updated second weight W2′ and the fourth weight W4, and then dividing it by two. Next, the updated third weight W3′ replaces the third weight W3 at the third time T3 to generate the updated urine volume data.

Similarly, the urine flow data processing method of the present invention is not only able to determine whether the difference between the updated second weight and the third weight is larger than the presetting threshold value, but also determine whether the difference between the updated third weight and the fourth weight is larger than the presetting threshold value at the same time. Furthermore, the urine flow data processing method of the present invention can determine the third weight and the fourth weight according to the different situations as mentioned above and further calculate the updated second weight, the third weight and the fourth weight. The methods and steps in different situations as mentioned above (such as situation that the second weight is adjusted by decreasing, by increasing or by continuous decreasing) are roughly the same as the aforementioned specific implementation methods, and will not be described again herein.

It should be noted that, it is able to further determine whether the difference between the updated third weight and the fourth weight is larger than the presetting threshold value, and then, further determine whether the difference between the updated fourth weight and the fifth weight is larger than the presetting threshold value, and so on, after step S7 in FIG. 5. Therefore, the urine flow data processing method of the present invention can iteratively determine and adjust the urine volume data one by one in ascending order of time to remove abnormal data caused by jitter phenomenon.

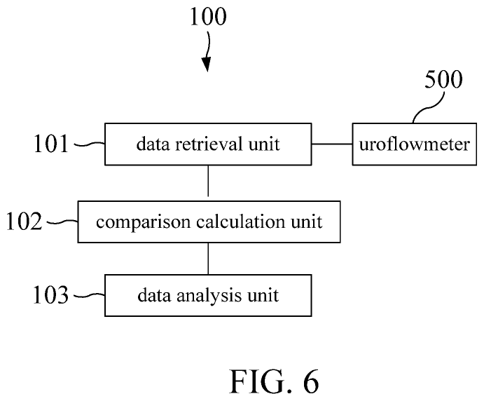

Please refer to FIG. 1 and FIG. 6. FIG. 6 is a functional block diagram illustrating the urine flow data processing system 100 according to an embodiment of the present invention. The steps of FIG. 1 can be executed by the system of FIG. 6. As shown in FIG. 6, in the present embodiment, the urine flow data processing system 100 comprises a data retrieval unit 101, a comparison calculation unit 102 and a data analysis unit 103. The data retrieval unit 101 is connected to the comparison calculation unit 102 and a uroflowmeter 500, and the comparison calculation unit 102 is connected to the data analysis unit 103. In practice, the data retrieval unit 101, the comparison calculation unit 102 and the data analysis unit 103 can be arranged in the same device or in different devices, and can be integrated into the same chip or one single chip.

In the present embodiment, the data retrieval unit 101 is configured to obtain the urine volume data measured by the uroflowmeter 500. In practice, the data retrieval unit 101 can be connected to the weight sensor of the uroflowmeter 500. When the weight sensor of the uroflowmeter 500 continues to measure the weight of the container, the data retrieval unit 101 can capture the weight by the weight sensor to obtain urine volume data.

In the present embodiment, the comparison calculation unit 102 pre-stores the presetting threshold value as mentioned above. The comparison calculation unit 102 is configured to calculate the difference between the first weight and the second weight, and compare the difference with the presetting threshold value. When the difference is larger than the presetting threshold value, the comparison calculation unit 102 generates the updated second weight according to the first weight, the second weight, and a third weight, and replaces the second weight with the updated second weight to generate the updated urine volume data.

In the present embodiment, the data analysis unit 103 is configured to analyze the updated urine volume data to generate a urine flow chart. In practice, the data analysis unit 103 can analyze the updated urine volume data through the filtering algorithm as mentioned above.

In the present embodiment, the comparison calculation unit 102 is configured to preprocess the urine volume data. After capturing the urine volume data from the uroflowmeter 500 by the data retrieval unit 101 (corresponding to step S1 in FIG. 1), the comparison calculation unit 102 determines the abnormal data caused by jitter phenomenon in the urine volume data (corresponding to step S2 in FIG. 1). Next, the comparison calculation unit 102 adjusts the abnormal data to generate the urine volume data which are reasonable and consistent with the actual urine or liquid (corresponding to step S3 in FIG. 1). Finally, the data analysis unit 103 analyzes the updated urine volume data to generate the urine flow curve (corresponding to step S4 in FIG. 1).

Furthermore, the urine flow data processing system 100 of FIG. 6 can also execute the steps of FIG. 3. The comparison calculation unit 102 determines whether the difference between the first weight and the second weight is larger than a presetting threshold value (step S2), and then determines whether the difference between the second weight and the third weight is larger than a presetting threshold value (step S5). Moreover, when the second weight is larger than or less than the first weight and the third weight, the comparison calculation unit 102 adds the first weight and the third weight together, and then divides the two to generate the updated second weight (step S31). In addition, when the second weight is less than the first weight and the third weight is less than the second weight, the comparison calculation unit 102 replaces the second weight and the third weight with the first weight (step S32).

Furthermore, the urine flow data processing system 100 of FIG. 6 can also execute the steps of FIG. 5. The comparison calculation unit 102 further determines whether the difference between the updated second weight and the third weight is larger than the presetting threshold value (step S6). When the result is yes, the comparison calculation unit 102 generates the updated third weight according to the updated second weight, the third weight and the fourth weight, and replaces the third weight with the updated third weight to generate the updated urine volume data (step S7).

In summary, the urine flow data processing method of the present invention can accurately determine the abnormal measurement data caused by the jitter phenomenon, and further reasonably adjust the abnormal measurement data according to the trend of the urine volume data. Furthermore, the urine flow data processing method of the present invention can iteratively determine and adjust urine volume data one by one in ascending order of time to remove abnormal data with jitter phenomenon, so that the urine flow chart generated from the urine volume data can be more in line with the actual situation, so as to improve the correctness and accuracy.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A urine flow data processing method, comprising the following steps of:
obtaining a urine volume data measured by a uroflowmeter, wherein the urine volume data comprises a first weight at a first time and a second weight at a second time, and the second time is later than the first time;
determining whether the difference between the first weight and the second weight is larger than a presetting threshold value;
generating an updated second weight corresponding to the second time according to the first weight, the second weight, and a third weight at a third time when the difference between the first weight and the second weight is larger than the presetting threshold value, and replacing the second weight with the updated second weight to generate an updated urine volume data, wherein the third time is later than the second time; and
analyzing the updated urine volume data to generate a urine flow chart.

2. The urine flow data processing method of claim 1, wherein the step of generating the updated second weight corresponding to the second time according to the first weight, the second weight, and a third weight when the difference between the second weight and the third weight is larger than the presetting threshold value, and replacing the updated second weight with the second weight to generate the updated urine volume data of the step, further comprises the following step of:
adding the first weight and the third weight together and then dividing the two to generate the updated second weight when the second weight is larger than or less than the first weight and the third weight, and replacing the second weight with the updated second weight to generate the updated urine volume data.

3. The urine flow data processing method of claim 2, further comprising the following step of:
replacing the second weight and the third weight with the first weight to generate the updated urine volume data when the second weight is less than the first weight and the third weight is less than the second weight.

4. The urine flow data processing method of claim 1, wherein when the difference between the updated second weight and the third weight is larger than the presetting threshold value, the urine flow data processing method further comprises the following step of:
generating an updated third weight corresponding to the third time according to the second weight, the third weight and a fourth weight at a fourth time, and replacing the third weight with the updated third weight to generate the updated urine volume data, wherein the fourth time is later than the second time.

5. The urine flow data processing method of claim 1, wherein the presetting threshold value is between 1 g and 1.5 g.

6. A urine flow data processing system, comprising:
a data retrieval unit, configured to obtain a urine volume data measured by a uroflowmeter, wherein the urine volume data comprises a first weight at a first time and a second weight at a second time, and the second time is later than the first time;
a comparison calculation unit, connected to the data retrieval unit and pre-stored a presetting threshold value, the comparison calculation unit is configured to calculate the difference between the first weight and the second weight, when the difference is larger than the presetting threshold value, the comparison calculation unit generates an updated second weight corresponding to the second time according to the first weight, the second weight, and a third weight at a third time, and replaces the second weight with the updated second weight to generate an updated urine volume data, wherein the third time is later than the second time; and
a data analysis unit, connected to the comparison calculation unit, the data analysis unit is configured to analyze the updated urine volume data to generate a urine flow chart.

7. The urine flow data processing system of claim 6, wherein the comparison calculation unit further calculates the difference between the second weight and the third weight, when the difference between the first weight and the second weight and the difference between the second weight and the third weight are both larger than the presetting threshold value, and also the second weight is larger than or less than the first weight and the third weight, the comparison calculation unit adds the first weight and the third weight together then divides the two to generate the updated second weight.

8. The urine flow data processing system of claim 7, when the second weight is less than the first weight and the third weight is less than the second weight, the comparison calculation unit replaces the second weight and the third weight with the first weight.

9. The urine flow data processing system of claim 6, wherein the comparison calculation unit further calculates the difference between the updated second weight and the third weight, when the difference is larger than the presetting threshold value, the comparison calculation unit generates an updated third weight corresponding to the third time according to the second weight, the third weight and a fourth weight at a fourth time, and the comparison calculation unit replaces the third weight with the updated third weight to generate the updated urine volume data, wherein the fourth time is later than the third time.

10. The urine flow data processing system of claim 7, wherein the presetting threshold value is between 1 g and 1.5 g.
